(19) United States
(12) Patent Application Publication
(10) Pub. No.: US 2021/0275040 A1
(43) Pub. Date: Sep. 9, 2021

(54) ULTRASOUND-BASED GUIDANCE FOR PHOTOACOUSTIC MEASUREMENTS AND ASSOCIATED DEVICES, SYSTEMS, AND METHODS

(71) Applicant: KONINKLIJKE PHILIPS N.V., Eindhoven (NL)

(72) Inventors: Jonathan Fincke, Cambridge, MA (US); Balasundar Iyyavu Raju, North Andover, MA (US); Jonathan Thomas Sutton, Boston, MA (US); Shriram Sethuraman, Lexington, MA (US)

(21) Appl. No.: 17/193,470

(22) Filed: Mar. 5, 2021

Related U.S. Application Data
(60) Provisional application No. 62/985,554, filed on Mar. 5, 2020.

(51) Int. Cl. A61B 5/026; A61B 5/00

(52) U.S. Cl. CPC A61B 5/026; A61B 5/489; A61B 5/7285; A61B 5/7425; A61B 5/0095

(57) ABSTRACT

Systems, devices, and methods for performing photoacoustic measurements using ultrasound-based guidance are provided. In one embodiment, an imaging system includes: an ultrasound imaging probe comprising an ultrasound transducer array, a processor circuit in communication with the ultrasound imaging probe, and a light source configured to emit light. The processor circuit receives first ultrasound data representative of an anatomical feature within a field of view, identifies a location of the anatomical feature within the field of view, and performs a photoacoustic measurement using the identified location of the anatomical feature. Performing the photoacoustic measurement includes: controlling the light source to emit light into the field of view and processing second ultrasound data representative of photoacoustic energy generated in the anatomical feature by the light source. The processor circuit then outputs a graphical representation of the photoacoustic measurement to a display.

### RELATED APPLICATION

The present application claims the benefit of and priority to Provisional Application Ser. No. 62/985,554, filed Mar. 5, 2020, which is incorporated herein in its entirety.

### TECHNICAL FIELD

The present disclosure relates generally to the acquisition and processing of ultrasound images and photoacoustic data. In particular, the present disclosure is directed to systems and methods for guiding a photoacoustic measurement procedure using ultrasound image data.

### BACKGROUND

Ultrasound imaging is frequently used to obtain images of internal anatomical structures of a patient. Ultrasound systems typically comprise an ultrasound transducer probe that includes a transducer array coupled to a probe housing. The transducer array is activated to vibrate at ultrasonic frequencies to transmit ultrasonic energy into the patient's anatomy, and then receive ultrasonic echoes reflected or backscattered by the patient's anatomy to create an image. Such transducer arrays may include various layers, including some with piezoelectric materials, which vibrate in response to an applied voltage to produce the desired pressure waves. These transducers may be used to successively transmit and receive several ultrasonic pressure waves through the various tissues of the body. The various ultrasonic responses may be further processed by an ultrasonic imaging system to display the various structures and tissues of the body.

Of recent interest is a form of ultrasound imaging that involves inducing acoustic vibrations in an anatomical feature using pulsed light waves, receiving or measuring the acoustic vibrations using an ultrasound transducer array, and computing a physiological measurement based on the received acoustic vibrations. This form of ultrasound imaging is referred to as photoacoustic imaging, and may beneficially provide for obtaining physiological measurements in a non-invasive manner. Photoacoustic imaging may be used to determine a variety of physiological parameters, including oxygen saturation of the blood vessels leading into an organ, hemoglobin concentration, and other parameters. For example, photoacoustic imaging may be used to measure the oxygen consumption of an organ of the body, such as the brain. Obtaining a photoacoustic measurement involves illuminating an anatomical feature of interest, such as a blood vessel, with sufficient intensity to induce acoustic vibrations that can be detected by the ultrasound transducer.

One of the central challenges in acquiring accurate photoacoustic measurements from human tissue is positioning the photoacoustic light source relative to the tissue volume of interest such that adequate acoustic signal can be received to make measurements. In that regard, the anatomical features of interest (e.g., blood vessel) may not be visible through the patient's skin such that a sonographer can properly place the light source. Another challenge with quantitative photoacoustic methods is selecting a photoacoustic waveform or region of interest in a photoacoustic image for analysis. Since many tissues absorb light and emit a photoacoustic signal, it can be difficult to assess which signal or portion of the signal came from the tissue region of interest.

### SUMMARY

The present disclosure describes systems, devices, and methods for performing photoacoustic measurements using ultrasound-based guidance. In one embodiment, an ultrasound-based photoacoustic measurement system includes an ultrasound transducer array positioned with respect to a photoacoustic light source and configured to obtain ultrasound data representative of an anatomical feature, such as a vessel. A processor circuit or processing system identifies a location of the anatomical feature based on the ultrasound data, and uses the identified location to guide the photoacoustic measurement. In some aspects, guidance may be automated, and may be provided in the form of control signals, user instructions, and/or image processing parameters. For example, the processor circuit may use a location of a vessel identified from ultrasound image data to set a spatial or temporal region of interest for processing photoacoustic signals. In another example, the processor circuit may provide instructions to a user or a control signal to an actuator to adjust a position and/or orientation of the light source of the photoacoustic subsystem to better illuminate the vessel, thereby increasing the strength of the resulting photoacoustic signals.

According to one embodiment of the present application, an imaging system includes: an ultrasound imaging probe comprising an ultrasound transducer array configured to emit ultrasound energy toward an anatomical feature within a field of view of the ultrasound transducer array; and a processor circuit in communication with the ultrasound imaging probe and a light source configured to emit light at an orientation with respect to the field of view. The processor circuit is configured to: receive first ultrasound data obtained by the ultrasound imaging probe, wherein the first ultrasound data is representative of the anatomical feature within the field of view; identify, by image processing of the first ultrasound data, a location of the anatomical feature within the field of view; and perform a photoacoustic measurement using the identified location of the anatomical feature within the field of view. Performing the photoacoustic measurement includes: controlling the light source to emit the light into the field of view; and processing second ultrasound data obtained by the ultrasound imaging probe, wherein the second ultrasound data is representative of photoacoustic energy generated in the anatomical feature by the light source. The processor circuit is further configured to output a graphical representation of the photoacoustic measurement to a display in communication with the processor circuit.

In some embodiments, the first ultrasound data comprises at least one of B-mode data or Doppler data. In some embodiments, the processor circuit is configured to identify the location of the anatomical feature using the B-mode data and the Doppler data. In some embodiments, the processor circuit is configured to: determine, based on the identified location of the anatomical feature, a gate for processing the second ultrasound data; and perform the photoacoustic measurement using the gate. In some embodiments, the gate comprises a temporal gate. In some embodiments, the gate comprises a spatial gate. In some embodiments, the first ultrasound data comprises Doppler data, and the processor circuit is configured to: determine a region of flow in the anatomical feature based on the Doppler data; and determine the spatial gate based on the region of flow. In some embodiments, the imaging system further includes an actuator coupled to the light source and configured to adjust at least one of a position or an orientation of the light source relative to the field of view of the ultrasound transducer array. In some embodiments, the actuator is communicatively coupled to the processor circuit. In some embodiments, the processor circuit is configured to control the actuator, based on the identified location of the anatomical feature, to adjust the at least one of the position or orientation of the light source relative to the field of view of the ultrasound transducer array. In some embodiments, the processor circuit is configured to control the actuator, based on the identified location of the anatomical feature, to advance the light source toward the anatomical feature such that tissue between the anatomical feature and the light source is deformed.

In some embodiments, the light source comprises a plurality of light elements positioned at different locations with respect to the ultrasound transducer array. In some embodiments, the processor circuit is configured to select, based on the identified location of the anatomical feature, one or more light elements of the plurality of light elements to perform the photoacoustic measurement. In some embodiments, the processor circuit is configured to: generate, using the identified location of the anatomical feature, a user instruction to reposition and least one of the light source or the ultrasound imaging probe; and output the user instruction to the display. In some embodiments, the processor circuit is configured to: generate a first image of the anatomical feature using the first ultrasound data; generate a second image of the anatomical feature using the second ultrasound data; and output the co-registered first and second images to the display.

In some embodiments, the processor circuit is configured to receive the first ultrasound data and the second ultrasound data at a same time. In some embodiments, the processor circuit is configured to receive the first ultrasound data and the second ultrasound data at different times in an interleaved fashion. In some embodiments, the imaging system further includes the light source. In some embodiments, the light source is coupled to the ultrasound imaging probe.

According to another embodiment of the present disclosure, a method for ultrasound imaging includes: receiving, by a processor circuit, first ultrasound data obtained by an ultrasound imaging probe comprising an ultrasound transducer array configured to emit ultrasound energy toward an anatomical feature within a field of view of the ultrasound transducer array, wherein the first ultrasound data is representative of the anatomical feature within the field of view; identifying, by image processing of the first ultrasound data, a location of the anatomical feature within the field of view; and performing a photoacoustic measurement using the identified location of the anatomical feature within the field of view. Performing the photoacoustic measurement includes: controlling a light source in communication with the processor circuit to emit light into the field of view at an orientation with respect to the field of view; and processing second ultrasound data obtained by the ultrasound imaging probe, wherein the second ultrasound data is representative of photoacoustic energy generated in the anatomical feature by the light source. The method further includes outputting a graphical representation of the photoacoustic measurement to a display in communication with the processor circuit.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of a photoacoustic measurement system, according to aspects of the present disclosure.

FIG. 2 is a diagrammatic view of an ultrasound transducer array and a photoacoustic subassembly performing a photoacoustic measurement, according to aspects of the present disclosure.

FIG. 3 is a diagrammatic view of an ultrasound transducer array and a photoacoustic subassembly performing a photoacoustic measurement, according to aspects of the present disclosure.

FIG. 4A is a combined B-mode and Doppler ultrasound image of a longitudinal cross-sectional view of a vessel, according to aspects of the present disclosure.

FIG. 4B is a photoacoustic image of a longitudinal cross-sectional view of a vessel, according to aspects of the present disclosure.

FIG. 5A is a combined B-mode and Doppler ultrasound image of a radial cross-sectional view of a vessel, according to aspects of the present disclosure.

FIG. 5B is a photoacoustic image of a radial cross-sectional view of a vessel, according to aspects of the present disclosure.

FIG. 6 is a graphical representation of a temporal gate applied to a photoacoustic waveform used to perform a photoacoustic measurement, according to aspects of the present disclosure.

FIG. 7A is a diagrammatic view of an ultrasound transducer array and a photoacoustic subassembly performing a photoacoustic measurement in which the vessel is not in the path of a beam of light, according to aspects of the present disclosure.

FIG. 7B is an ultrasound image of a vessel obtained using the ultrasound transducer array and the photoacoustic subassembly in the configuration shown in FIG. 8A, according to aspects of the present disclosure.

FIG. 7C is a photoacoustic image of the vessel obtained using the ultrasound transducer array and the photoacoustic subassembly in the configuration shown in FIG. 8A, according to aspects of the present disclosure.

FIG. 8A is a diagrammatic view of the ultrasound transducer array and the photoacoustic subassembly of FIG. 8A performing a photoacoustic measurement in which the vessel is in the path of the beam of light, according to aspects of the present disclosure.

FIG. 8B is an ultrasound image of a vessel obtained using the ultrasound transducer array and the photoacoustic subassembly in the configuration shown in FIG. 9A, according to aspects of the present disclosure.

FIG. 8C is a photoacoustic image of the vessel obtained using the ultrasound transducer array and the photoacoustic subassembly in the configuration shown in FIG. 9A, according to aspects of the present disclosure.

FIG. 9 is a top plan view of an ultrasound transducer array of an ultrasound probe including a plurality of photoacoustic light elements, according to aspects of the present disclosure.

FIG. 10 is a top plan view of an ultrasound transducer array of an ultrasound probe including a plurality of photoacoustic light elements, according to aspects of the present disclosure.

FIG. 11 is a flow diagram of a method for performing a photoacoustic measurement using ultrasound-based guidance, according to aspects of the present disclosure.

FIG. 12 is a flow diagram of a method for performing a photoacoustic measurement using ultrasound-based guidance, according to aspects of the present disclosure.

FIG. 13 is a schematic diagram of a processor circuit, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

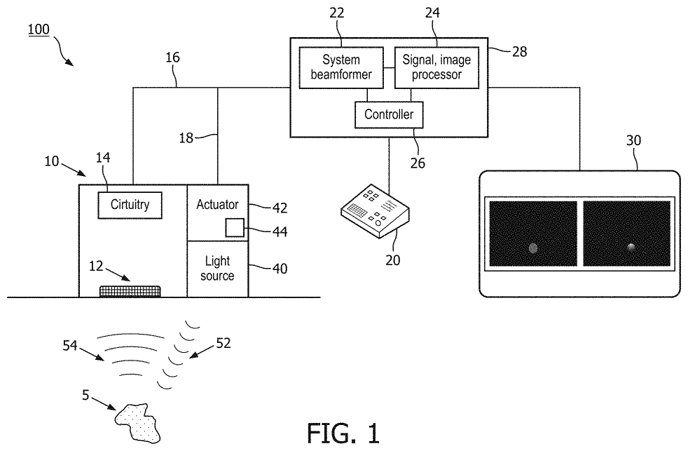

In FIG. 1, an ultrasound-based photoacoustic measurement system 100 according to embodiments of the present disclosure is shown in block diagram form. In some aspects, the photoacoustic measurement system 100 includes devices and/or subsystems for ultrasound imaging, such as an ultrasound probe 10 having a transducer array 12 comprising a plurality of ultrasound transducer elements or acoustic elements. In some instances, the array 12 may include any number of acoustic elements. For example, the array 12 can include between 1 acoustic element and 100000 acoustic elements, including values such as 2 acoustic elements, 4 acoustic elements, 36 acoustic elements, 64 acoustic elements, 128 acoustic elements, 300 acoustic elements, 812 acoustic elements, 3000 acoustic elements, 9000 acoustic elements, 30,000 acoustic elements, 65,000 acoustic elements, and/or other values both larger and smaller. In some instances, the acoustic elements of the array 12 may be arranged in any suitable configuration, such as a linear array, a planar array, a curved array, a curvilinear array, a circumferential array, an annular array, a phased array, a matrix array, a one-dimensional (1D) array, a 1.X dimensional array (e.g., a 1.5D array), or a two-dimensional (2D) array. The array of acoustic elements (e.g., one or more rows, one or more columns, and/or one or more orientations) can be uniformly or independently controlled and activated. The array 12 can be configured to obtain one-dimensional, two-dimensional, and/or three-dimensional images of patient anatomy. In some embodiments, the ultrasound probe 10 includes a single transducer element, such as a mechanically-scanned transducer element.

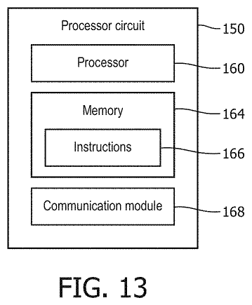

Referring again to FIG. 1, the acoustic elements of the array 12 may comprise one or more piezoelectric/piezoresistive elements, lead zirconate titanate (PZT), piezoelectric micromachined ultrasound transducer (PMUT) elements, capacitive micromachined ultrasound transducer (CMUT) elements, and/or any other suitable type of acoustic elements. The one or more acoustic elements of the array 12 are in communication with (e.g., electrically coupled to) electronic circuitry 14. In some embodiments, such as the embodiment of FIG. 1, the electronic circuitry 14 can comprise a microbeamformer (μBF). In other embodiments, the electronic circuitry comprises a multiplexer circuit (MUX). The electronic circuitry 14 is located in the probe 10 and communicatively coupled to the transducer array 12. In some embodiments, one or more components of the electronic circuitry 14 can be positioned in the probe 10. In some embodiments, one or more components of the electronic circuitry 14, can be positioned in a computing device or processing system 28. The computing device 28 may be or include a processor, such as one or more processors in communication with a memory. As described further below, the computing device 28 may include a processor circuit as illustrated in FIG. 13. In some aspects, some components of the electronic circuitry 14 are positioned in the probe 10 and other components of the electronic circuitry 14 are positioned in the computing device 28. The electronic circuitry 14 may comprise one or more electrical switches, transistors, programmable logic devices, or other electronic components configured to combine and/or continuously switch between a plurality of inputs to transmit signals from each of the plurality of inputs across one or more common communication channels. The electronic circuitry 14 may be coupled to elements of the array 12 by a plurality of communication channels. The electronic circuitry 14 is coupled to a cable 16, which transmits signals including ultrasound imaging data to the computing device 28.

In the computing device 28, the signals are digitized and coupled to channels of a system beamformer 22, which appropriately delays each signal. The delayed signals are then combined to form a coherent steered and focused receive beam. System beamformers may comprise electronic hardware components, hardware controlled by software, or a microprocessor executing beamforming algorithms. In that regard, the beamformer 22 may be referenced as electronic circuitry. In some embodiments, the beamformer 22 can be a system beamformer, such as the system beamformer 22 of FIG. 1, or it may be a beamformer implemented by circuitry within the ultrasound probe 10. In some embodiments, the system beamformer 22 works in conjunction with a microbeamformer (e.g., electronic circuitry 14) disposed within the probe 10. The beamformer 22 can be an analog beamformer in some embodiments, or a digital beamformer in some embodiments. In the case of a digital beamformer, the system includes anaolog-to-digital converters which convert analog signals from the array 12 into sampled digital echo data. The beamformer 22 generally will include one or more microprocessors, shift registers, and or digital or analog memories to process the echo data into coherent echo signal data. Delays are effected using various techniques such as by the time of sampling of received signals, the write/read interval of data temporarily stored in memory, or by the length or clock rate of a shift register as described in U.S. Pat. No. 4,173,007 to McKeighen et al., the entirety of which is hereby incorporated by reference herein. Additionally, in some embodiments, the beamformer can apply appropriate weight to each of the signals generated by the array 12. The beamformed signals from the image field are processed by a signal and image processor 24 to produce 2D or 3D images for display on an image display 30. The signal and image processor 24 may comprise electronic hardware components, hardware controlled by software, or a microprocessor executing image processing algorithms. It generally will also include specialized hardware or software which processes received echo data into image data for images of a desired display format such as a scan converter. In some embodiments, beamforming functions can be divided between different beamforming components. For example, in some embodiments, the system 100 can include a microbeamformer located within the probe 10 and in communication with the system beamformer 22. The microbeamformer may perform preliminary beamforming and/or signal processing that can reduce the number of communication channels required to transmit the receive signals to the computing device 28.

Control of ultrasound system parameters such as scanning mode (e.g., B-mode, Doppler, M-mode), probe selection, beam steering and focusing, and signal and image processing is done under control of a system controller 26 which is coupled to various modules of the system 100. The system controller 26 may be formed by application specific integrated circuits (ASICs) or microprocessor circuitry and software data storage devices such as RAMs, ROMs, or disk drives. In the case of the probe 10, some of this control information may be provided to the electronic circuitry 14 from the computing device 28 over the cable 16, conditioning the electronic circuitry 14 for operation of the array as required for the particular scanning procedure. The user inputs these operating parameters with a user interface device 20.

In some embodiments, the image processor 24 is configured to generate images of different modes to be further analyzed or output to the display 30. For example, in some embodiments, the image processor can be configured to compile a B-mode image, such as a live B-mode image, of an anatomy of the patient. In other embodiments, the image processor 24 is configured to generate or compile a Doppler image, such as a color Doppler or Power Doppler image. A doppler image can be described as an image showing moving portions of the imaged anatomy.

It will be understood that the computing device 28 may comprise hardware circuitry, such as a computer processor, application-specific integrated circuit (ASIC), field-programmable gate array (FPGA), capacitors, resistors, and/or other electronic devices, software, or a combination of hardware and software. In some embodiments, the computing device 28 is a single computing device. In other embodiments, the computing device 28 comprises separate computer devices in communication with one another.

Further, it will be understood that although the present disclosure refers to synthetic aperture external ultrasound imaging using an external ultrasound probe, one or more aspects of the present disclosure can be implemented in any suitable ultrasound imaging probe or system, including external ultrasound probes and intraluminal ultrasound probes. For example, aspects of the present disclosure can be implemented in ultrasound imaging systems using a mechanically-scanned external ultrasound imaging probe, an intracardiac (ICE) echocardiography catheter and/or a transesophageal echocardiography (TEE) probe, a rotational intravascular ultrasound (IVUS) imaging catheter, a phased-array IVUS imaging catheter, a transthoracic echocardiography (TTE) imaging device, or any other suitable type of ultrasound imaging device.

In some aspects, the system 100 may be used to obtain photoacoustic measurements and/or images. In that regard, the system 100 further comprises a photoacoustic subsystem or subassembly that includes a light source 40 and an actuator 42 mechanically coupled to the light source 40. The light source is configured to emit a beam 52 of light toward an anatomical feature 5, which may comprise a blood vessel. The beam 52 may be pulsed to induce acoustic (e.g., ultrasonic) vibrations the anatomical feature 5. In some embodiments, the probe 10, light source 40, and actuator 42 form an integral unit coupled to and/or positioned within a housing. In some embodiments, the light source 40 and the actuator 42 are coupled to the probe 10 via an attachment such that the light source 40 and/or the actuator 42 may be coupled to an existing commercially-available probe. In that regard, in some aspects, the light source 40 and the actuator 42 may be part of a photoacoustic subsystem of the system 100. The light source 40 is maintained at a position and orientation relative to the transducer array 12. In some aspects, the position and orientation of the light source 40 may be referred to as a pose. In that regard, the path of the beam 52 of light emitted by the light source 40 may be changed by adjusting the pose of the light source 40.

In the illustrated embodiment, the light source 40 and actuator 42 are communicatively coupled to the computing device 28 via a cable 18. In some embodiments, the light source 40 and the actuator 42 are coupled to the computing device 28 via separate cables. In some aspects, the controller 26 may be configured to control the probe 10, actuator 42, and light source 40. In some embodiments, the controller 26 comprises separate controller units dedicated to each of the probe 10, the light source 40, and the actuator 42. Further, in the illustrated embodiment, the actuator 42 includes a feedback sensor 44 configured to detect or monitor actuation of the light source 40 by the actuator 42. For example, in some embodiments, the feedback sensor 44 is configured to detect a position and/or orientation of the light source 40 relative to the ultrasound transducer array 12. In some embodiments, the feedback sensor 44 is configured to detect a position and/or orientation of the light source 40 relative to the patient (e.g., the anatomical feature 5, the skin, etc.) In some embodiments, the feedback sensor 44 is configured to detect a force applied to the patient's skin by the light source 40, and/or to detect an amount of deformation of the patient's skin or anatomy by the light source 40. Accordingly, the feedback sensor 44 may be used by the controller 26 in controlling the actuator 42 using a feedback loop (e.g., a proportional-integral-derivative (PID) loop). In some embodiments, the feedback sensor is configured to measure displacement of the light source, force experienced by the moving subsystem, and/or displacement or collapse of the blood vessel under interrogation. In some embodiments, the processor circuit may utilize that information to control the amount of movement of the actuator and/or light source, and to adapt the photoacoustic signal processing based on the information from one of the above measurements.

The system 100 is configured to perform a photoacoustic measurement procedure to determine one or more physiological characteristics of an anatomical structure, such as a blood vessel. In an exemplary embodiment, the photoacoustic measurement procedure includes activating the light source 40 to emit the beam 52 of light into the body of a patient to induce photoacoustic vibrations in the anatomical feature 5. The vibrations cause acoustic waves 54 to propagate through the tissue to the transducer array 12, which receives the acoustic waves 54 and converts them into an electrical signal. Physiological characteristics of the anatomical feature 5, such as oxygen concentration or hemoglobin concentration, can be inferred from the magnitude and/or frequency composition of the received acoustic signals. In an exemplary embodiment, the anatomical feature 5 comprises a blood vessel, such as a vein or artery. Photoacoustic measurements can be used to determine oxygen concentration of the blood flowing into and/or out of an organ of the body, such as the brain, to determine the oxygen consumption of the organ.

As described above, beams of light 52 from the light source 40 attenuate exponentially in the tissue. Accordingly, it is desirable to not only position and orient the light source 40 such that the anatomical feature is within the path of the beam 52, but also to position and orient the light source 40 to reduce or minimize the distance between the light source 40 and the anatomical feature. However, in some instances, placing the light source 40 to obtain a reliable photoacoustic measurement can be a difficult and imprecise process. For example, many blood vessels are not externally visible. Further, even when the anatomical feature 5 is within the path of the beam 52 and reasonably close to the light source 40, processing the photoacoustic data may involve significant amounts of error, as the light source 40 may induce photoacoustic vibrations in the tissue and other features within the tissue that are not of interest for the photoacoustic measurement. Accordingly, the present disclosure provides systems, methods, and devices for leveraging information obtained using ultrasound imaging techniques (e.g., B-mode image data and/or Doppler image data) to guide photoacoustic measurement procedures.

FIG. 13 is a schematic diagram of a processor circuit 150, according to embodiments of the present disclosure. The processor circuit 150 may be implemented in the computing device 28, the signal and image processor 24, the controller 26, and/or the probe 10 of FIG. 1. As shown, the processor circuit 150 may include a processor 160, a memory 164, and a communication module 168. These elements may be in direct or indirect communication with each other, for example via one or more buses.

The processor 160 may include a central processing unit (CPU), a digital signal processor (DSP), an ASIC, a controller, an FPGA, another hardware device, a firmware device, or any combination thereof configured to perform the operations described herein. The processor 160 may also be implemented as a combination of computing devices, e.g., a combination of a DSP and a microprocessor, a plurality of microprocessors, one or more microprocessors in conjunction with a DSP core, or any other such configuration.

The memory 164 may include a cache memory (e.g., a cache memory of the processor 160), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 164 includes a non-transitory computer-readable medium. The memory 164 may store instructions 166. The instructions 166 may include instructions that, when executed by the processor 160, cause the processor 160 to perform the operations described herein with reference to the processor 28 and/or the probe 10 (FIG. 1). Instructions 166 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 168 can include any electronic circuitry and/or logic circuitry to facilitate direct or indirect communication of data between the processor 28, the probe 10, and/or the display 30. In that regard, the communication module 168 can be an input/output (I/O) device. In some instances, the communication module 168 facilitates direct or indirect communication between various elements of the processor circuit 150 and/or the processing system 106 (FIG. 1).

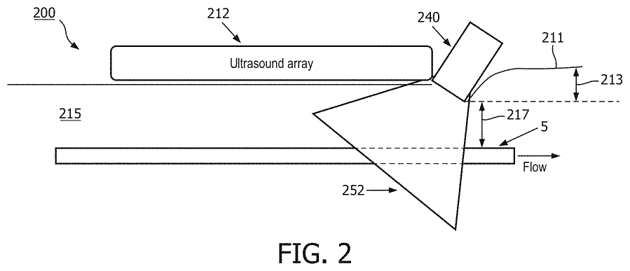

FIG. 2 is a diagrammatic view of a photoacoustic measurement system 200 obtaining a photoacoustic measurement, according to aspects of the present disclosure. In that regard, the system 200 includes an ultrasound transducer array 212 comprising a plurality of ultrasound transducer elements configured to obtain ultrasound image data and/or photoacoustic data from the body of the patient. The system 200 further includes a light source 240 configured to emit a beam of light 252 toward a blood vessel 5 within the tissue 215. In FIG. 2, the imaging plane or field of view of the ultrasound transducer array 212 is parallel with the longitudinal axis of the vessel 5 to obtain a longitudinal cross-sectional view of the vessel 5. The light source 240 is shown slightly pressed into the skin surface 211 of the tissue 215. As described further below, in some aspects, it may be advantageous to press the light source 240 into the tissue 215 by deforming the skin 211 of the patient to reduce the path length 217 between the light source 240 and the vessel 5. For example, in some embodiments, the light source 240 is movable such that it can be pressed into the skin surface 211 by a displacement distance 213 to reduce the path length 217 between the light source 240 and the vessel 5. In some embodiments, the system 200 further includes a feedback sensor configured to measure displacement of the moving subsystem, force experienced by the moving subsystem, and/or displacement or collapse of the blood vessel under interrogation. The processor circuit may be configured to utilize these measurements to adapt the photoacoustic signal processing.

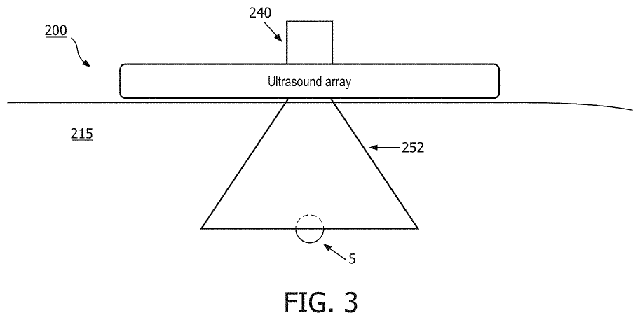

FIG. 3 shows the system 200 shown in FIG. 2 with the ultrasound transducer array 212 positioned such that the imaging plane or field of view is perpendicular to the blood vessel 5 to obtain a radial cross-sectional view of the vessel 5. In some aspects, the light source 240 may be considered optimally placed and oriented with respect to the vessel 5 to obtain a photoacoustic measurement in both FIG. 2 and FIG. 3. However, even when optimally placed, it may be desirable to identify specific regions or portions of a photoacoustic image corresponding to the vessel 5 to obtain a photoacoustic image. Since many tissues and anatomical structures within a given field of view of the ultrasound transducer absorb light and emit a photoacoustic signal, it can be difficult to assess which signals came from the tissue region of interest (e.g., the vessel 5), and which signals came from tissues that are not of interest. Accordingly, the present disclosure provides for ultrasound-based guidance for identifying portions of photoacoustic signals and/or images to analyze for photoacoustic measurements. By using ultrasound images to localize the tissue region of interest, one or more gates can be determined for processing the photoacoustic image or signal, thereby focusing the photoacoustic measurement on the regions more likely to yield accurate photoacoustic measurements.

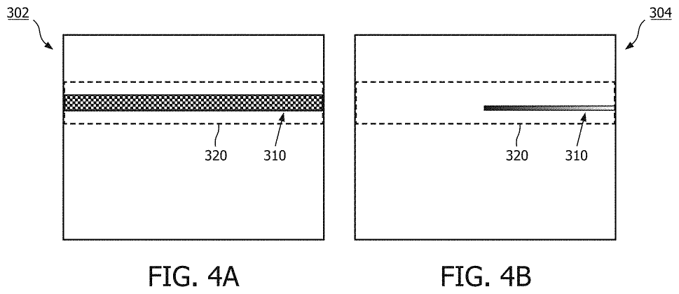

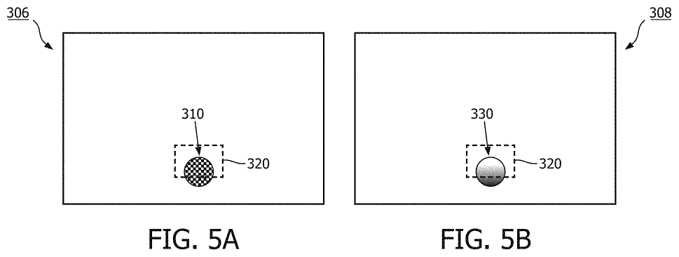

FIGS. 4A-5B show ultrasound images 302, 306 and photoacoustic images 304, 308 obtained using the system 200 shown in FIGS. 2 and 3. In that regard, the images 302, 304 of FIGS. 4A and 4B are obtained according to configuration shown in FIG. 2 in which the field of view of the ultrasound transducer array 212 is parallel to the vessel 5. FIG. 4A is a combined B-mode and Doppler image 302 obtained using the first field of view, and FIG. 4B is a photoacoustic image 304 obtained using the first field of view. The images 306, 308 of FIGS. 5A and 5B are obtained according to configuration shown in FIG. 3 in which the field of view of the ultrasound transducer array 212 is perpendicular to the vessel 5. FIG. 5A is a combined B-mode and Doppler image 306 obtained using the second field of view, and FIG. 5B is a photoacoustic image 308 obtained using the second field of view.

As shown in FIG. 4A, the combined image includes B-mode information representative of a vessel wall of the vessel 310, and Doppler information representative of flowing blood within the vessel 310. The B-mode information of the vessel wall is shown as the white outlines of the vessel 310, and the Doppler information is shown as the patterned interior portion of the vessel 310. In some embodiments, the Doppler information is used to localize coarse region of flow in the vessel initially. This coarse information could feed back to a beamforming unit to perform high resolution (e.g., line density, low F #) B-mode or harmonic imaging to determine much higher resolution echogenicity changes indicating the proximal vessel wall. By determining the boundaries of the vessel wall, a spatial gate 320 can be computed that corresponds to a detected location of the vessel 310 in the image. Using this gate 320, the processor circuit analyzes the region of the photoacoustic image 304 within the gate 320 to more readily identify relevant portions of the photoacoustic image 304 of the vessel 310, as shown in FIG. 4B. In some aspects, the gate 320 determined from the ultrasound image 302 may be used as a search region 320 in the corresponding photoacoustic image 304. In some aspects, using the gate 320 obtained by the ultrasound data may improve the accuracy and/or efficiency of the photoacoustic measurement.

The gate 320 may be determined or computed to focus on a proximal region of the vessel 310 closer to the ultrasound transducer. In some aspects, the photoacoustic signals may be stronger in the portion of the vessel 310 that is closer to the ultrasound transducer. In other embodiments, the gate is determined or computed to include an entirety of the vessel 310. While the gate 320 shown in FIGS. 4A-5B is rectangular, it will be understood that the gate 320 may comprise other shapes, such as a polygonal shape, circular shape, elliptical shape, irregular shape, or any other suitable shape or combinations thereof. For example, in some embodiments, the shape of the gate 320 matches a determined shape of a vessel feature, such as the vessel 310 lumen or the vessel wall.

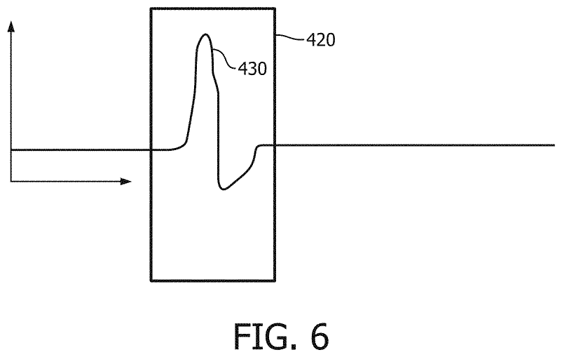

In some embodiments, the system 200 is configured to determine a temporal gate for the photoacoustic signals, rather than a spatial gate. In that regard, FIG. 6 shows a temporal gate 420 determined using B-mode and/or Doppler image data of a vessel, as applied to a photoacoustic signal 430. Similar to the spatial gate 320 shown in FIGS. 4A-5B, the temporal gate 420 may isolate a portion of the photoacoustic signal 430 used to obtain a photoacoustic measurement.

In some instances, it may be challenging to properly position and orient a light source of a photoacoustic measuring system to illuminate the vessel of interest. For example, the blood vessel may not be externally visible to the physician. Accordingly, the present disclosure provides for ultrasound-based guidance for photoacoustic light source placement. As described further below, an imaging system may use ultrasound image data (e.g., B-mode and/or Doppler data) of an anatomical feature, in addition to a known position and/or orientation of the light source relative to the ultrasound transducer array to adjust a position and/or orientation of the light source to direct more light to the anatomical feature.

In some embodiments, guidance is output by the system in the form of a user instruction to adjust a position of an ultrasound probe and/or light source. The user instruction may be output to a display, speaker, and/or other user interface device. In some embodiments, guidance is output as a computer command to an actuator configured to mechanically adjust a pose (i.e. position and/or orientation) of the light source relative to the ultrasound transducer array. For example, the actuator may comprise an electric motor, gears, rack and pinion, servo motor, hinge, and/or other mechanical components coupled to the light source and configured to adjust the pose of the light source in one or more degrees of freedom. In some embodiments, the actuator is controllable to by a processor circuit to automatically perform a motorized adjustment of the pose of the light source. In some embodiments, the actuator is manually controllable by a user to adjust the pose of the light source. Accordingly, in some embodiments, the light source is movable by the actuator in a manner so as to reduce the distance between a blood vessel and the light source. The actuator may allow for translation along the surface of the skin as well as the capability to deform the skin surface such that the light source is made to be closer to the vessel of interest. In other embodiments, the light source may include a plurality of light source elements (e.g., optical fibers or bundles of optical fibers) that can be selectively activated according to the instructions output by a guidance system.

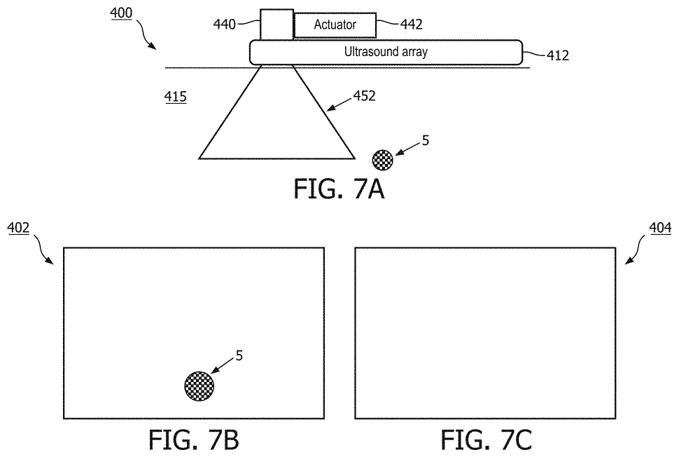

FIG. 7A is a diagrammatic view of a photoacoustic measurement system 400, according to an embodiment of the present disclosure. The system 400 includes an ultrasound transducer array 412 configured to be positioned with respect to a patient to obtain ultrasound image data of the anatomy of a patient, including a vessel 5 and tissue 415. The system 400 further includes a light source 440 co-located with the ultrasound transducer array and configured to illuminate the vessel 5 and/or tissue 415. The system 400 further includes an actuator 442 or actuator assembly coupled to the light source 440 and configured to adjust a pose of the light source 440 (and therefore, a path of the beam 452 of light) relative to the ultrasound transducer array 412. FIG. 7B is an ultrasound image 402 representative of the vessel 5 and obtained by the ultrasound transducer array 412 as shown in FIG. 7A. Specifically, FIG. 7B is a combined B-mode and Doppler image 402 of a radial cross-sectional view of the vessel 5. FIG. 7C is a photoacoustic image 404 obtained using the same field of view shown in FIG. 7B and with the light source 440 positioned relative to the ultrasound transducer array 412 and the vessel 5 as shown in FIG. 7A. Referring to FIG. 7B, the vessel 5 is depicted within the field of view of the transducer array 412. By contrast, the vessel 5 is not shown in the photoacoustic image 404 of FIG. 7C because, although the vessel 5 is within the field of view of the transducer array 412, the vessel 5 is not within the beam path 452 of the light source 440. Accordingly, any photoacoustic energy generated by the vessel 5 is not detected by the ultrasound transducer array 412.

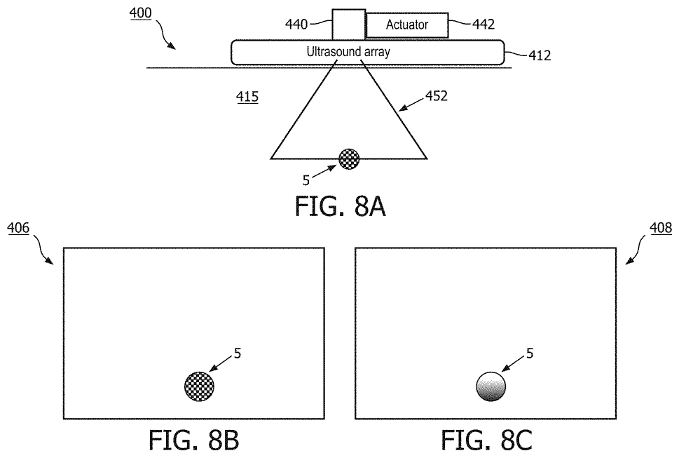

FIG. 8A is a diagrammatic view of the photoacoustic measurement system 400 shown in FIG. 7A, with the pose of the light source 440 adjusted relative to the transducer array 412 such that the beam 452 illuminates the vessel 5. FIG. 8B shows a combined B-mode/Doppler image 406 obtained with the transducer array 412 positioned as in FIG. 8A. In that regard, the image 406 of FIG. 8B is substantially the same as in FIG. 7B because the transducer array 412 has not moved relative to the vessel 5. However, the photoacoustic image 408 shown in FIG. 8C now shows the vessel 5, as the light source is positioned such that a sufficient amount of the beam 452 illuminates the vessel 5 to perform a photoacoustic measurement. In some embodiments, the vessel 5 is shown in the same position in the ultrasound image 406 and the photoacoustic image 408 because the same field of view is used to obtain both images 406, 408. In other embodiments, the vessel 5 is shown in different locations and/or in different sizes in the respective images 406, 408. For example, different fields of view of the transducer array 412 may be used to obtain the ultrasound image 406 and the photoacoustic image 408.

The actuator 442 shown in FIGS. 7A and 8A may be in communication with a controller or processor circuit configured to generate control signals for the actuator 442 based on a location of the vessel 5 detected based on ultrasound image data. For example, the processor circuit may determine a location of the vessel 5 within the field of view by image processing the ultrasound image data. Based on the determined location and a known position and/or orientation of the light source 440 with respect to the field of view of the ultrasound transducer array 412, the processor circuit computes a movement to position the light source 440 such that the beam 452 illuminates the vessel 5. The processor circuit then generates a control signal to activate the actuator 442 to carry out the computed movement. In some embodiments, the actuator 442 comprises one or more of an electric motor, a servo motor, gears, a rack and pinion, pneumatic devices, springs, magnets, hinges, pistons, and/or any other suitable actuating mechanism controllable by the processor circuit to adjust the pose of the light source 440. In some embodiments, the photoacoustic measurement system 400 does not include a controllable actuator, but includes a mechanical coupling assembly that can be manually adjusted by an operator to change the position and/or orientation of the light source 440 with respect to the ultrasound transducer array 412.

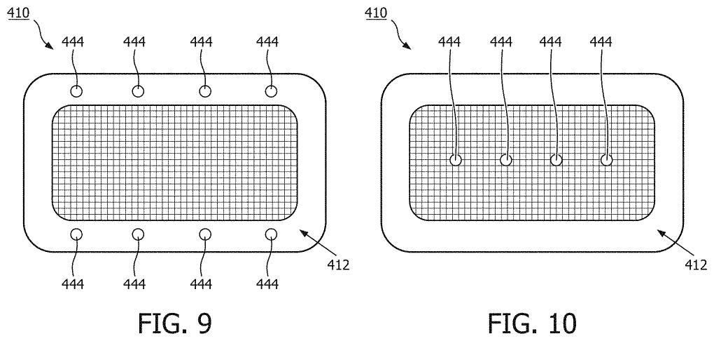

As shown in FIGS. 9 and 10, in some embodiments, an ultrasound probe 410 may include a light source having a plurality of individual light elements 444 positioned at different locations with respect to the ultrasound transducer array. The light elements 444 can be selectively activated by the processor circuit based on the determined location of the vessel and the known position and/or orientation of the light elements 444 with respect to the field of view of the transducer array 412. In some embodiments, the light elements 444 are co-located with the transducer array 412 on the probe 410. For example, in the embodiment shown in FIG. 9, the light elements 444 are disposed around a periphery of the ultrasound transducer array 412 on a surface of the probe 410. In the embodiment shown in FIG. 10, the light elements 444 are positioned within the transducer array 412.

The light source 440 and/or light elements 444 may comprise, for example, one or more optical fibers, light-emitting diodes, lasers, incandescent light bulbs, fluorescent bulbs, or any other suitable type of light element. Further, the light source 440 may include lenses, mirrors, prisms, or other optical components configured to direct light to a location (e.g., a vessel) and/or to control characteristics of the light, such as frequency, bandwidth, focus, or other characteristics. In some embodiments, the light source may be configured to emit light having a frequency profile that includes multiple frequencies or frequency peaks. For example, the frequency profile may include frequencies associated with a photoacoustic response of blood and/or tissue. In some embodiments, the frequency profile includes one or more frequencies in the infrared (IR) and/or near-infrared (NIR) spectrum. In some embodiments, the frequency profile includes one or more frequencies between 500 nm and 1100 nm. In some embodiments, the frequency profile includes one or more frequencies or frequency peaks centered at approximately (i.e., +/−10%) 600 nm, 700 nm, 800 nm, 900 nm, and/or 1050 nm.

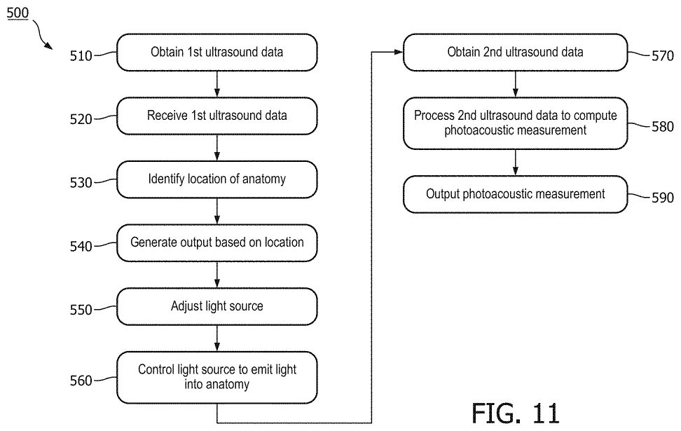

FIG. 11 is a flow diagram illustrating a method 500 for performing a photoacoustic measurement using ultrasound-based guidance, according to an embodiment of the present disclosure. It will be understood that the method 500 may be performed using the devices and/or systems described above, such as the system 100 shown in FIG. 1, including the ultrasound probe 10, the light source 40, the actuator, the computing device 28, and/or the display 30. In step 510, an ultrasound transducer array obtains first ultrasound data representative of an anatomical feature within a field of view of the ultrasound transducer array. In that regard, the first ultrasound data may be representative of a blood vessel. The first ultrasound data may be obtained when a user, such as a sonographer or physician, places the transducer array of an ultrasound probe against the skin of the patient proximate a vessel of interest to emit ultrasound energy toward the vessel. In some instances, the sonographer may desire to obtain photoacoustic images and/or measurements that can be used to determine oxygen consumption of a patient's organ, such as the patient's brain. Accordingly, the sonographer may place the ultrasound probe against the patient's neck at a location proximate a vessel leading into or away from the brain, such as a carotid artery, a vertebral artery, occipital artery, and/or any other suitable vessel.

In step 520, the processor receives the first ultrasound data. The first ultrasound data may be used to generate B-mode and/or Doppler data (e.g., power Doppler, color Doppler, etc.). In that regard, in some embodiments, the first ultrasound data is obtained by interleaving B-mode image sequences and Doppler imaging sequences. In some embodiments, the ultrasound transducer array provides ultrasound signals or data, and a processor circuit generates B-mode image data and Doppler image data based on the same ultrasound signals. In some embodiments, only B-mode image data is generated. In other embodiments, only Doppler data is generated. In some embodiments, the processor circuit receives the first ultrasound data from the ultrasound imaging probe. In some embodiments, the processor circuit receives or retrieves the first ultrasound data from a memory device.

In step 530, the processor circuit identifies, by image processing of the first ultrasound data, a location of the vessel within the field of view of the ultrasound transducer array. In some embodiments, the processor circuit generates ultrasound image data, such as B-mode image data and/or Doppler data using the first ultrasound data, and performs image processing on the B-mode and/or Doppler data to identify the location of the vessel within the field of view. In some embodiments, the processor circuit uses the Doppler data as seed points and B-mode image data to determine one or more boundaries of the vessel such as the inner diameter. Image processing may include one or more of erosion, dilation, segmentation, border detection, and/or any other suitable morphological or image processing technique to identify an anatomical feature in the ultrasound data. Additional information regarding morphological processing techniques can be found in, for example, U.S. Patent Application Publication No. 2017/0273658 titled “Acoustic streaming for fluid pool detection and identification” filed Aug. 12, 2015 with Shougang Wang et al. as inventors, U.S. Patent Application Publication No. 2014/0334680 titled “Image processing apparatus,” filed Nov. 14, 2012 with Iwo Willem Oscar Serile et al., as inventors, and Shawn Lankton, et al., “Localizing Region-Based Active Countrs,” IEEE Transactions on Image Processing, Vol. 17, No. 11 (November 2008), each of which is hereby incorporated by reference in its entirety.

In step 540, the processor circuit generates an output based on the identified location of the vessel. The output may be generated based on the identified location of the vessel and a known position and/or orientation of the light source with respect to the field of view of the ultrasound transducer array. In some embodiments, the pose of the light source relative to the ultrasound transducer array may be fixed. In other embodiments, the pose of the light source relative to the ultrasound transducer array is adjustable. In some embodiments, the pose is manually adjustable by a user. In other embodiments, the light source is mechanically coupled to an actuator configured adjust the pose of the light source. In that regard, in some embodiments, the output generated in step 540 includes a control signal for controlling the actuator to adjust the pose of the light source. In some embodiments, the control signal is received by an electric motor (e.g., stepper motor), a servo motor, pneumatic control valve, and/or any other suitable actuator component configured to adjust the pose of the light source. In some embodiments, the output indicates which of a plurality of individual light elements to activate to illuminate the vessel. In some embodiments, the output is sent to a display and includes an indicator instructing a user to adjust a position of the ultrasound probe and/or the light source. For example, the indicator may include a textual instruction, a graphical instruction, and/or an audible instruction. The instruction may relate to a translation, tilt, fan, sweep, compression, or any other suitable type of movement of the ultrasound probe and/or the light source to direct light from the light source to the vessel.

In step 550, the light source is adjusted based on the output. In some embodiments, the pose of the light source is automatically adjusted by the processor circuit and the actuator to illuminate the vessel. In some embodiments, the light source is coupled to an ultrasound probe at a fixed pose, position, and/or orientation relative to the ultrasound transducer array. In some embodiments, the pose, position, and/or orientation of the light source is manually adjusted by the user according to instructions output to a display, speaker, and/or other interface device. For example, in some embodiments, the light source movable in a manner so as to reduce the distance between a blood vessel and the light source. In some embodiments, a movable component allows translation along the surface of the skin as well as the capability to deform the skin surface such that the optical source is made to be closer to the vessel of interest. In some embodiments, the pose, position, and/or orientation of the light source may be adjusted by the user by following on-screen instructions associated with the output generated in step 540 to adjust the pose, position, and/or orientation of the ultrasound probe. For example, the light source may be coupled to the ultrasound probe by a jig or attachment that is sized, shaped, and structurally arranged to be coupled to the ultrasound probe. The jig or attachment may be configured to be attached to an existing or commercially-available ultrasound probe. The light source may also be coupled to the jig or attachment. In other embodiments, the light source and the ultrasound probe form an integral device including a single housing sized, shaped, and structurally arranged to be grasped by the hand of a user. In some embodiments, the light source is separate from the ultrasound probe and/or manually repositionable relative to the ultrasound probe. Accordingly, in some embodiments, the pose of the light source may be adjusted manually by a user independently of the pose, position, and/or orientation of the ultrasound probe. In some embodiments, the processor circuit determines, based on the identified location of the vessel in the field of view, that the current pose, position, and/or orientation of the light source is acceptable or optimal, and no adjustment of the light source is performed. Whether the pose of the light source is adjusted by an actuator or manually by moving the probe, the output may instruct the adjustment to place a vessel of interest within the optical path of the beam of light of the light source and/or improve the signal-to-noise ratio (SNR) of the photoacoustic signals from the vessel.

In some embodiments, adjusting the position and/or orientation of the light source may include pressing the light source into the skin of the patient to reduce the distance between the light source and the anatomical feature (e.g., vessel) of interest. Accordingly, in some embodiments, the actuator is configured to cause the light source to deform the skin of the patient by advancing the light source toward the anatomical feature. In some embodiments, the planar position and orientation of the light source is first adjusted based on image data, and then the light source is pressed into the skin along the axis of the light source. In some embodiments, the position adjustment and pressing movement are performed simultaneously. In some embodiments, the adjustments are carried out by the actuator automatically based on input from image processing and/or from other sensors or feedback devices. In that regard, in some embodiments, a feedback sensor is communicatively coupled to the light source and is configured to detect or measure one or more aspects associated with the movement (e.g., force, position), which is used as feedback to control the actuator. For example, the feedback sensor may include a load sensor, an encoder (e.g., rotary encoder), linear variable differential transformer (LVDT), hall effect sensor, proximity sensor, or any other suitable type of sensor capable of measuring the position and/or orientation of the light source relative to the ultrasound probe or transducer array. Controlling the actuation or movement of the light source may include using a feedback loop with the output of the feedback sensor and an input. For example, a PID loop may be used to control the actuation. In some embodiments, the processor circuit is further configured to detect using, for example, image processing of the image data and/or photoacoustic data, that the vessel of interest has collapsed, indicating excessive force applied by the light source. In response to detecting the collapse or deformation of the vessel, the processor circuit may be configured to adjust the force applied by the actuator on the light source. Further, the processor circuit may be configured to change photoacoustic signal processing parameters based on information obtained by the feedback sensor and/or the ultrasound imaging data.

In some embodiments, the movement of the light source may be performed manually by a user. For example, the light source may be separate or separable from the ultrasound probe, and the processor circuit may be configured to generate and output user instructions to move the light source (e.g., translation, tilt, compression into the skin) based on the output generated in step 540. In some embodiments, the light source is movably coupled to the ultrasound probe by a jig, brace, or other attachment that allows for movement in one or more degrees of freedom. In some embodiments, the system further includes an acoustic coupling fluid dispensing subassembly configured to dispense an acoustic coupling gel in response to detecting insufficient contact between the ultrasound transducer and the skin of the patient. For example, the processor circuit may be configured to perform image processing on the image data obtained by the ultrasound transducer to detect poor acoustic coupling between the transducer and the skin of the patient. Based on this detection, the processor circuit may dispense acoustic coupling fluid at or near the ultrasound transducer to improve acoustic coupling. Accordingly, the fluid dispensing subassembly may ensure acoustic coupling after movement of the ultrasound probe and/or light source wherein the fluid or acoustic coupling gel is made to cover a void if created by the movement.

In some aspects, controlling the light source to reduce the distance to the vessel of interest may reduce the photoacoustic path length and allow for photoacoustic measurements to be made from blood vessels that are deep within the tissue under the skin surface. In some instances, for example, a physician may desire to obtain blood oxygenation measurements using the photoacoustic techniques described herein to determine an amount of oxygenated perfusion to the brain. For example, the techniques described herein may be used to obtain photoacoustic measurements from the internal jugular vein, which provide an indicator of oxygenated perfusion to the brain.

In step 560, the processor circuit controls the light source to emit light into the anatomy. In some embodiments, the light source comprises a laser, such as a Helium Neon, Argon, Krypton, or Xenon Ion, Yttrium Aluminum Garnet (YAG), Semiconductor Diode, Diode, and/or any other suitable type of laser. The laser may be configured to operate in one or more modes, including continuous wave (CW), single pulsed, single pulsed Q-switched, mode-locked, repetitively pulsed, and/or any other suitable mode. In other embodiments, the light source comprises an incandescent bulb, a diode, such as a light-emitting diode (LED), fluorescent bulb, halogen bulb, or any other suitable source. In some embodiments, one or more optical fibers are coupled to a light element (e.g., laser, light bulb) and configured to deliver light within the field of view of the ultrasound transducer array. In some embodiments, the light source is co-located with the ultrasound transducer array. The light may comprise light or electromagnetic energy in the IR spectrum, NIR spectrum, microwave spectrum, visible spectrum, ultraviolet (UV) spectrum, or any other spectrum suitable to induce acoustic vibrations in the vessel.

In step 570, with the light source activated to direct light to the vessel, second ultrasound data, such as photoacoustic data, is obtained using the ultrasound transducer array. The second ultrasound data or photoacoustic data is representative of the acoustic vibrations induced by the light source. In some embodiments, a photoacoustic measuring device includes distinct ultrasound transducers or transducer arrays to receive the first ultrasound data and the second ultrasound data, respectively. In some embodiments, the second ultrasound data is obtained at a same time as the first ultrasound data. In that regard, in some embodiments, the processor circuit is configured to generate ultrasound image data (e.g., B-mode data, Doppler data) in addition to photoacoustic data from the same ultrasound signals obtained by the ultrasound transducer array. In other embodiments, the first ultrasound data is obtained at a different time than the second ultrasound data. In some embodiments, the second ultrasound data is obtained in a sequence that interleaves the acquisition of the first ultrasound data and the second ultrasound data.

In step 580, the processor circuit processes the second ultrasound data or photoacoustic data to compute a photoacoustic measurement. In some embodiments, computing the photoacoustic measurement includes generating a photoacoustic image based on the second ultrasound data. Computing the photoacoustic measurement may include performing a spectral or frequency analysis on the second ultrasound data. For example, computing the photoacoustic measurement may include comparing the intensity of one frequency band or bands to another frequency band or bands. In some embodiments, computing the photoacoustic measurement includes comparing a magnitude of the second ultrasound data to a local energy deposition. In some embodiments, computing the photoacoustic measurement includes analyzing dual optical wavelength photoacoustic waveforms detected by an ultrasound transducer array.

In step 590, the processor circuit outputs a graphical representation of the computed photoacoustic measurement to a display or interface device. The photoacoustic measurement may include an oxygen saturation, oxygen concentration, and/or hemoglobin concentration value. The photoacoustic measurement may be associated with oxygenated hemoglobin (HbO₂) and/or deoxygenated hemoglobin (Hb). In some embodiments, a photoacoustic image may be output to a display along with the graphical representation of the photoacoustic measurement. In some embodiments, the photoacoustic image is co-registered with a corresponding ultrasound image generated based on the first ultrasound data. For example, the processor circuit may be configured to output, to the display, a combined B-mode and Doppler image, alongside a photoacoustic image. The ultrasound image and the photoacoustic image may be obtained using an interleaved sequence in which ultrasound and photoacoustic image streams are received by the processor and output to show respective real-time or live views of the vessel.

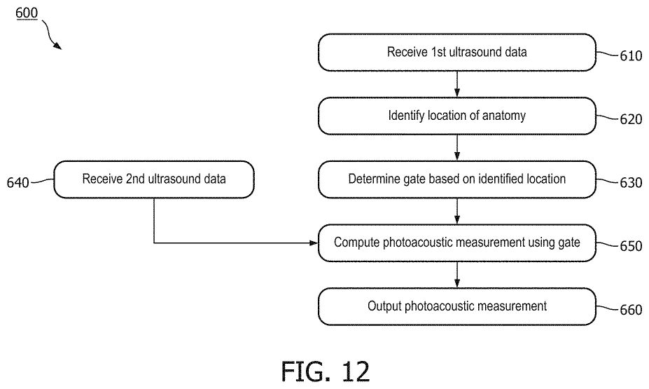

It may be beneficial, in some instances, to gate photoacoustic measurements to specific areas or a photoacoustic image or specific time windows in a photoacoustic signal. Gating may increase the accuracy and/or efficiency of the photoacoustic measurement by the processor circuit. Gating may be used in addition to the approaches (e.g., method 500) described above with respect to adjusting the position of the light source, or independently of the adjustment of the light source. FIG. 12 illustrates a method 600 for computing a photoacoustic measurement using a gate determined using ultrasound image data, according to some embodiments of the present disclosure. It will be understood that the method 600 may be performed using one or more of the devices, systems, and/or methods described above, such as the system 100 shown in FIG. 1.

In Step 610, the processor circuit receives first ultrasound data obtained by an ultrasound transducer array. The first ultrasound data may be used to generate B-mode and/or Doppler data (e.g., power Doppler, color Doppler, etc.). In that regard, in some embodiments, the first ultrasound data is obtained by interleaving B-mode and Doppler imaging sequences. In some embodiments, the ultrasound transducer array provides ultrasound signals or data, and a processor circuit generates B-mode image data and Doppler image data based on the same ultrasound signals. In some embodiments, only B-mode image data is generated. In other embodiments, only Doppler data is generated. In some embodiments, the processor circuit receives the first ultrasound data from the ultrasound imaging probe. In some embodiments, the processor circuit receives or retrieves the first ultrasound data from a memory device.

In step 620, the processor circuit identifies, by image processing of the first ultrasound data, a location of the vessel within the field of view of the ultrasound transducer array. In some embodiments, the processor circuit generates ultrasound image data, such as B-mode image data and/or Doppler data using the first ultrasound data, and performs image processing on the B-mode and/or Doppler data to identify the location of the vessel within the field of view. In some embodiments, the processor circuit uses the Doppler data as seed points and B-mode image data to determine one or more boundaries of the vessel such as the inner diameter. Image processing may include one or more of erosion, dilation, segmentation, border detection, and/or any other suitable image processing technique to identify an anatomical feature in the ultrasound data.

In step 630, the processor circuit determines a gate based on the identified location of the vessel. In some embodiments, the gate comprises a spatial gate specifying a region in which the vessel is located within the ultrasound image and/or photoacoustic image. Embodiments of spatial gates are shown in FIGS. 4A-5B. In other embodiments, the gate comprises a temporal gate indicating a time window for processing ultrasound signals of the second ultrasound data, as shown in FIG. 6, for example. In one embodiment, power- or color-Doppler imaging is used to localize coarse flow regions in the vessel initially. This coarse information is then fed back to a beamforming unit to perform high resolution (e.g. line density, low F #) b-mode or harmonic imaging to determine much higher resolution echogenicity changes indicating the proximal vessel wall. In step 640, the processor circuit receives second ultrasound data, or photoacoustic data. The second ultrasound data or photoacoustic data is representative of the acoustic vibrations induced by the light source. In some embodiments, a photoacoustic measuring device includes distinct ultrasound transducers or transducer arrays to receive the first ultrasound data and the second ultrasound data, respectively. In some embodiments, the second ultrasound data is obtained at a same time as the first ultrasound data. In that regard, in some embodiments, the processor circuit is configured to generate ultrasound image data (e.g., B-mode data, Doppler data) in addition to photoacoustic data from the same ultrasound signals obtained by the ultrasound transducer array. In some embodiments, the second ultrasound data is obtained in a sequence that interleaves the acquisition of the first ultrasound data and the second ultrasound data. In some embodiments, the processor circuit automatically detects and localizes the vessel of interest using a Doppler image and a B-mode image generated based on the first ultrasound data, and a photoacoustic image generated based on the second ultrasound data. The processor circuit then sets the a priori analysis region in the photoacoustic image and/or photoacoustic waveforms of the second ultrasound data.

In step 650, the processor circuit computes a photoacoustic measurement using the second ultrasound data and the gate determined or computed in step 630. In step 660, the processor circuit outputs the photoacoustic measurement to the display. The photoacoustic measurement may be output as an oxygen saturation, oxygen concentration, and/or hemoglobin concentration. The photoacoustic measurement may be associated with oxygenated hemoglobin (HbO₂) and/or deoxygenated hemoglobin (Hb). In some embodiments, a photoacoustic image may be output to a display along with the photoacoustic measurement. In some embodiments, the photoacoustic image is co-registered with a corresponding ultrasound image generated based on the first ultrasound data. For example, the processor circuit may be configured to output, to the display, a combined B-mode and Doppler image, alongside a photoacoustic image. In some embodiments, a graphical representation of the gate determined in step 630 is also output to the display. In some embodiments, only one of the ultrasound image generated using the first ultrasound data or the photoacoustic image generated using the second ultrasound data is output to the display.

It will be understood that one or more of the steps of the methods 500, 600 described above may be performed by one or more components of an ultrasound imaging system, such as a processor or processor circuit, a multiplexer, a beamformer, a signal processing unit, an image processing unit, or any other suitable component of the system. For example, one or more steps described above may be carried out by the processor circuit 150 described with respect to FIG. 13. The processing components of the system can be integrated within the ultrasound imaging device, contained within an external console, contained within a separate component, and/or distributed in various hardware components between the ultrasound imaging device, the external console, and/or the separate component.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An imaging system, comprising:
an ultrasound imaging probe comprising an ultrasound transducer array configured to emit ultrasound energy toward an anatomical feature within a field of view of the ultrasound transducer array; and
a processor circuit in communication with the ultrasound imaging probe and a light source configured to emit light at an orientation with respect to the field of view, wherein the processor circuit is configured to:
receive first ultrasound data obtained by the ultrasound imaging probe, wherein the first ultrasound data is representative of the anatomical feature within the field of view;
identify, by image processing of the first ultrasound data, a location of the anatomical feature within the field of view;
perform a photoacoustic measurement using the identified location of the anatomical feature within the field of view, wherein performing the photoacoustic measurement includes:
controlling the light source to emit the light into the field of view; and
processing second ultrasound data obtained by the ultrasound imaging probe, wherein the second ultrasound data is representative of photoacoustic energy generated in the anatomical feature by the light source; and
output a graphical representation of the photoacoustic measurement to a display in communication with the processor circuit.

2. The system of claim 1, wherein the first ultrasound data comprises at least one of B-mode data or Doppler data, and wherein the processor circuit is configured to identify the location of the anatomical feature using the B-mode data and the Doppler data.

3. The system of claim 1, wherein the processor circuit is configured to:
determine, based on the identified location of the anatomical feature, a gate for processing the second ultrasound data; and
perform the photoacoustic measurement using the gate.

4. The system of claim 3, wherein the gate comprises a temporal gate.

5. The system of claim 3, wherein the gate comprises a spatial gate.

6. The system of claim 5, wherein the first ultrasound data comprises Doppler data, and wherein the processor circuit is configured to:
determine a region of flow in the anatomical feature based on the Doppler data; and
determine the spatial gate based on the region of flow.

7. The system of claim 1, further comprising an actuator coupled to the light source and configured to adjust at least one of a position or an orientation of the light source relative to the field of view of the ultrasound transducer array.

8. The system of claim 7, wherein the actuator is communicatively coupled to the processor circuit, and wherein the processor circuit is configured to control the actuator, based on the identified location of the anatomical feature, to adjust the at least one of the position or orientation of the light source relative to the field of view of the ultrasound transducer array.

9. The system of claim 8, wherein the processor circuit is configured to control the actuator, based on the identified location of the anatomical feature, to advance the light source toward the anatomical feature such that tissue between the anatomical feature and the light source is deformed.

10. The system of claim 1, wherein the light source comprises a plurality of light elements positioned at different locations with respect to the ultrasound transducer array, and wherein the processor circuit is configured to select, based on the identified location of the anatomical feature, one or more light elements of the plurality of light elements to perform the photoacoustic measurement.

11. The system of claim 1, wherein the processor circuit is configured to:
generate, using the identified location of the anatomical feature, a user instruction to reposition and least one of the light source or the ultrasound imaging probe; and
output the user instruction to the display.

12. The system of claim 1, wherein the processor circuit is configured to:
generate a first image of the anatomical feature using the first ultrasound data;
generate a second image of the anatomical feature using the second ultrasound data; and
output the co-registered first and second images to the display.

13. The system of claim 1, wherein the processor circuit is configured to receive the first ultrasound data and the second ultrasound data at a same time.

14. The system of claim 1, wherein the processor circuit is configured to receive the first ultrasound data and the second ultrasound data at different times in an interleaved fashion.

15. The system of claim 1, further comprising the light source.

16. The system of claim 15, wherein the light source is coupled to the ultrasound imaging probe.

17. A method for ultrasound imaging, comprising:
receiving, by a processor circuit, first ultrasound data obtained by an ultrasound imaging probe comprising an ultrasound transducer array configured to emit ultrasound energy toward an anatomical feature within a field of view of the ultrasound transducer array, wherein the first ultrasound data is representative of the anatomical feature within the field of view;
identifying, by image processing of the first ultrasound data, a location of the anatomical feature within the field of view;
performing a photoacoustic measurement using the identified location of the anatomical feature within the field of view, wherein performing the photoacoustic measurement includes:
controlling a light source in communication with the processor circuit to emit light into the field of view at an orientation with respect to the field of view; and
processing second ultrasound data obtained by the ultrasound imaging probe, wherein the second ultrasound data is representative of photoacoustic energy generated in the anatomical feature by the light source; and
outputting a graphical representation of the photoacoustic measurement to a display in communication with the processor circuit.
